(19) United States
(12) Patent Application Publication
(10) Pub. No.: US 2008/0051413 A1
(43) Pub. Date: Feb. 28, 2008

(54) Nitrophenylpiperazine derivative of xanthine which relaxes tracheal airway and increases respiratory performance

(75) Inventor: Ing-Jun Chen, KaoHsiung City (TW)

Correspondence Address: TROJAN LAW OFFICES, 9250 WILSHIRE BLVD, SUITE 325, BEVERLY HILLS, CA, 90212, US

(73) Assignee: KAOHSIUNG MEDICAL UNIVERSITY

(21) Appl. No.: 11/510,213

(22) Filed: Aug. 25, 2006

(51) Int. Cl. A61K 31/522; C07D 473/02

(52) U.S. Cl. 514/252.16; 544/276

(57) ABSTRACT

The effects of KMUP-3, (7-[2-[4-(4-Nitrobezene)peperazinyl]ethyl]-1,3-dimethylxanthine) on tracheal smooth muscle are provided. Intratracheal instillation of KMUP-3 avoids bronchoconstriction, decreases of lung resistance, and increases of dynamic lung compliance. These merits provide insightful benefits for the treatment in asthma and COPD (chronic obstructive pulmonary disease).

### FIELD OF THE INVENTION

The present invention relates to a nitrophenylpiperazine derivative of xanthine, and more particularly to one which relaxes tracheal airway and increases respiratory performance.

### BACKGROUND OF THE INVENTION

Xanthine derivatives, including theophylline, have long been recognized as bronchodilators because of their nonspecific inhibition activities of phosphodiesterase (PDE) to increase cAMP, and they have been widely used in the therapy of bronchospastic diseases, including acute asthmatic attacks, emphysema, and chronic bronchitis, which are often associated with immunoresponses (Caramori G and Adcock 1 (2003) Pharmacology of airway inflammation in asthma and COPD. Pulm Pharmacol Ther 16:247-277). The immunopharmacologic effects of xanthine derivatives have recently become a particular subject of interest (Kobayashi M, Nasuhara Y, Betsuyaku T, Shibuya E, Tanino Y, Tanino M, Takamura K, Nagai K, Hosokawa T, and Nishimura M (2004) Effect of low-dose theophylline on airway inflammation in COPD, Respirology 9:249-254). Nevertheless, several important drawbacks remain, and a low therapeutic margin of safety, associated with a pharmacokinetic profile highly influenced by individual factors, results in the need for monitoring blood levels, which makes the drug hard to use widely in clinics (Barnes P J (2003) Theophylline: new perspectives for an old drug. Am J Respir Crit Care Med 167:813-818).

Likewise, the properties of xanthine derivatives to increase epithelium-dependent cGMP and protein kinase G (PKG) expression, which suppress tumor necrosis factor-_(TNF-_)-induced response in tracheal smooth muscle cells (TSMCs), have not been explored thoroughly. TNF-_ has been described to up-regulate 5-hydroxytryptamine_{2A}-mediated and B1/B2 receptor responses in airway inflammation (Adner M. Rose A C, Zhang Y, Sward K, Benson M, Uddman R, Shankley N P, and Cardell L O (2002). An assay to evaluate the long-term effects of inflammatory mediators on murine airway smooth muscle: evidence that TNFalpha up-regulates 5-HT(2A)-mediated contraction. Br J Pharmacol 137:971-982; Zhang Y, Adner M, and Cardell L O (2005) Glucocorticoids suppress transcriptional up-regulation of bradykinin receptors in a murine in vitro model of chronic airway inflammation. Clin Exp Allergy 35:531-538), to modulate tracheal responsiveness to G-protein-coupled receptor agonist (Chen H, Tliba O, Van Besien C R, Panettieri R A Jr, and Amrani Y (2003) TNF-[alpha] modulates murine tracheal rings responsiveness to G-protein-coupled receptor agonists and KCl. J Appl Physiol 95:864-872), and to act as an inflammatory cytokine involved in asthmatics (Leung T F, Wong G W, Ko F W, Li C Y, Yung E, Lam C W, and Fok T F (2005) Analysis of growth factors and inflammatory cytokines in exhaled breath condensate from asthmatic children, Int Arch Allergy Immunol 137:66-72). Previously, KMUP-1 has been stated to possess airway relaxation activities (Wu B N, Lin R J, Lo Y C, Shen K P, Wang C C, Lin Y T, and Chen I J (2004) KMUP-1, a xanthine derivatives, induces relaxation of guinea-pig isolated trachea; the role of the epithelium, cyclic nucleotides and K⁺ channels. Br J Pharmacol 142:1105-1114). To create a more hopeful xanthine-based tracheal smooth muscle (TSM) relaxant, KMUP-3 (FIG. 1) was synthesized (Wu B N, Chen I C, Lin C Y, Chiu C C, An L M, and Chen I J (2005) Aortic smooth muscle relaxants KMUP-3 and KMUP-4, two nitrophenylpiperazine derivatives of xanthine, display cGMP enhancing activity: roles of endothelium, phosphodiesterase and K⁺-channel. J Cardiovase Pharmacol 46:600-608) and investigated regarding its mechanism of TSM-relaxing activity and the benefits of its intratracheal administration. In routine clinical practice, in addition to corticosteroids and _{—}₂-adrenoreceptor agonist inhalants, however, no xanthine derivatives are inhaled for the treatment of bronchocontraction or airway inflammation.

It has been reported that epithelial cells release various smooth muscle inhibitory mediators, such as nitric oxide (NO) and prostaglandin E₂, as well as the so-called epithelium-derived relaxing factor detected by the coaxial bioassay system (Nijkamp F P, van der Linde H J, and Folkerts G (1993) Nitric oxide synthesis inhibitors induce airway hyperresponsiveness in the guinea pig in vivo and in vitro: role of the epithelium. Am Rev Respir Dis 148:727-734; Folkerts G and Nijkamp F P (1998) Airway epithelium: more than just a barrier! Trends Pharmacol Sci 19:334-341). Some reports regarding xanthine derivatives on tracheal epithelium, modulated by NO, have been published (Kanoh S, Kondo M, Tamaoki J, Kobayashi H, Motoyoshi K, and Nagai A (2000) Effects of reactive oxygen species on intracellular calcium in bovine tracheal epithlium: modulation by nitric oxide. Exp Lung Res 26:335-348; Vajner L, Konradova V, Uhlik J, and Zocova J (2002) The effects of intravenously administered methylxanthine preparations on the glycoconjugate composition of goblet cells in rabbit tracheal epithelium. Acta Histochem 104:107-112).

Because the effects of nitrophenylpeperaxine derivative of xanthine on the airway smooth muscle are not clear, the present inventor has invented this invention after many experiments and detailed studies are carried out. Thus, how to rectify the foresaid conventional drawback is the main purpose of the present invention.

### SUMMARY OF THE INVENTION

It is an object of the present invention to provide a nitrophenylpeperazine derivative xanthine being one of a substrate with the formula I and a pharmaceutically acceptable salt thereof and having an ability of relaxing airway smooth muscle

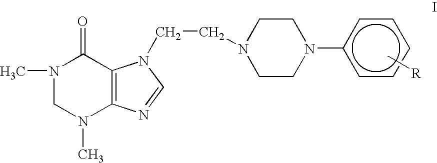

### Wherein R is a nitroso group.

Preferably, the nitrophenylpiperazine derivative of xanthine has an effect of tracheal relaxation.

Preferably, the nitrophenylpeperazine derivative of xanthine has an effect of anti-airway constriction induced by a tumor necrosis factor (TNF)-_.

Preferably, the nitrophenylpeperazine derivative of xanthine provides the benefits for the treatment of asthma and COPD (chronic obstructive pulmonary disease).

According to one aspect of the present invention, there is provided a pharmaceutical composition for relaxing airway smooth muscle comprising an above nitrophenylpiperazine derivative of xanthine and a pharmaceutically acceptable carrier.

In accordance with the present invention, the pharmaceutical composition further comprises an excipient.

In accordance with another aspect of the present invention, there is provided a method of manufacturing a medicament for relaxing airway smooth muscle comprising providing one of a nitrophenylpiperazine derivative of xanthine having the formula I and a pharmaceutically acceptable salt thereof as a raw material

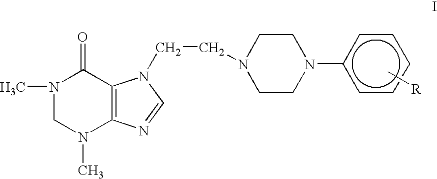

wherein R is a nitroso group.

In accordance with another aspect of the present invention, there is provided a use of a nitrophenylpiperazine derivative of above formula I for manufacturing a medicament having an ability of relaxing airway smooth muscle.

The pharmaceutically acceptable salt called in the present invention indicates the salt thereof is used to contact tissues of human beings or animals without generating a excessively toxic, inflammatory, allergic reaction or other similar diseases. The salt thereof is prepared when the compound of the present invention is separated or purified, or after a reaction between a suitable organic acid and a base.

The excipient called in the present invention indicates a filler, packing agent, diluent or any other form of auxiliary formula of nontoxic inert solid, semisolid or liquid. When it is needed, a dying agent, releasing agent, coating agent, sweetening agent, fragrance or fragrant agent, preservative agent or anti-oxidant is added.

The foregoing and other features and advantages of the present invention will be more clearly understood through the following descriptions with reference to the drawings, wherein:

### BRIEF DESCRIPTION OF THE DRAWINGS

FIG. 1 is a drawing showing the chemical structures of the KMUP-3 and KMUP-1;

FIG. 2 is a drawing showing R_{L} and C_{dyn} effects of KMUP-3 on TNF-_-induced (0.01 mg/kg) bronchoconstriction by intratracheal instillation according to the present invention;

FIG. 3(A) is a drawing showing effects of KMUP-3 on carbacol (1 _M)-precontracted guinea pig trachea in the E_{P}⁺ and epithelium-denuded (E_{P}⁻) tissues compared with the E_{P}⁺ (ANOVA followed by Turkey test) according to the present invention;

FIG. 3(B) is a drawing showing effects of KMUP-3, theophylline, IBMX, milrinonve, rolipram, zaprinast, and leveromakalin (100 _M) on carbachol (1 _M)-precontracted guinea pig epithelium-intact trachea according to the present invention;

FIG. 4(A)-4(E) are drawings showing effects of KMUP-3 on carbachol (1 _M)-precontracted guinea pig epithelium-intact trachea in the absence or presence of arious potassium channel blockers according to the present invention;

FIG. 5 is a drawing showing effects of KMUP-3 on carbachol (1 _M)-precontracted guinea pig epithelial-intact trachea in the absence or presence of L-NAME (100 _M), ODQ (1 _M), or SQ 22536 (100 _M) compared with the control according to the present invention;

FIG. 6 is a drawing showing effects of KMUP-3, milrinone, rolipram, zaprinast, theophylline, and aminophylline (100 _M) on cGMP and cAMP levels in guinea pig tracheal smooth muscle cells compared with the vehicle according to the present invention;

FIG. 7(A) is a drawing showing representative western blots and the corresponding group data depicting PKA_{RI} and PKG₁_{_{—}}₁_{_{—}} protein expression in cultured guinea pig tracheal smooth muscle cells incubated for 30 min in the absence (vehicle) and presence of 8-Br-cAMP (1 _M), SQ 22536 (100 _M), SQ 22536+KMUP-3 (1 _M), or KMUP-3 (1 _M) according to the present invention;

FIG. 7(B) is a drawing showing representative western blots and the corresponding group data depicting PKA_{RI} and PKG₁_{_{—}}₁_{_{—}} protein expression in cultured guinea pig tracheal smooth muscle cells incubated for 30 min in the absence (vehicle) and presence of 8-Br-cGMP (1 _M), ODQ (10 _M), ODQ+KMUP-3, or KMUP-3, SQ 22536 or ODQ was treated 30 min prior to KMUP-3 according to the present invention; and

FIG. 8 is a drawing showing a proposed mechanism of action of KMUP-3 on the intracellular cGMP and cAMP synthesis and metabolism, K⁺ channel opening, and phosphodiesterase inhibition.

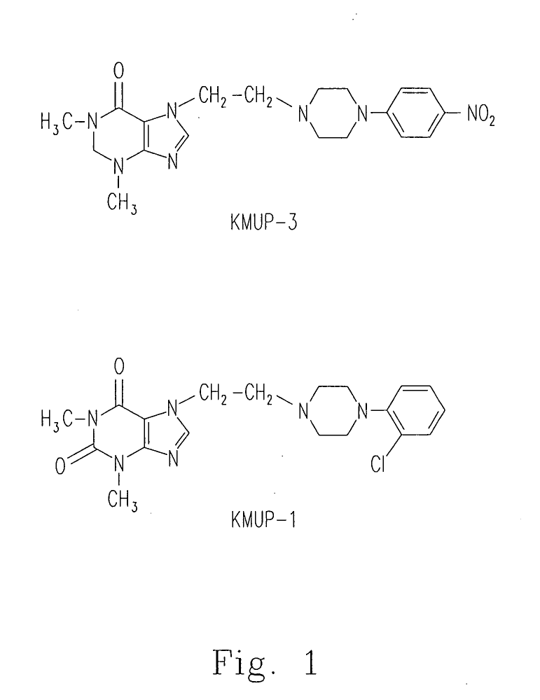

### DETAILED DESCRIPTION OF THE PREFERRED EMBODIMENT

In the present invention, we characterize the effects of KMUP-3 on TSM, associated specific epithelium-dependent protein kinase pathways, and opening of K⁺-channels. The combination of epithelium-derived action (including NO release), PDE inhibition, sGC stimulation, and K⁺-channel opening activity is suggested to enhance the results achieved. Additive effects of those pathways result in increase of cGMP/cAMP, coexpression of PKG/protein kinase A (PKA), and protection activity against TNF-_-induced bronchoconstriction.

Animals: Male Dunkin Harley guinea pigs (350-450 g) were provide by the National Laboratory Animal Breeding and Research Center (Taipei, Taiwan) and housed under conditions of constant temperature and controlled illumination. Food and water were available and libitum. The study was approved by the Animal Care and Use Committee of the Kaohsiung Medical University.

Pulmonary Function: Guinea pegs (350-450 g) were anesthetized with pentobarbital (40 mg/kg i.p.) and maintained with further i.v. doses of 2 to 5 mg/kg when required according to methods previously described (Wu B N, Lin R J, Lo Y C, Shen K P, Wang C C, Lin Y T, and Chen I J (2004) KMUP-1, a xanthine derivative, induces relaxation of guinea-pig isolated trachea: the role of the epithelium, cyclic nucleotides and K⁺ channels, Br J Pharmacol 142:1105-1114). The animal was tethered in a supine position, and the trachea was cannulated below the larynx with a short tracheal cannula via a tracheotomy. Tracheal pressure was measured by a catheter connected to the side arm of the tracheal cannula. The carotid artery and jugular vein were cannulated to monitor blood pressure and heart rate and for drug administration, respectively. Throughout the experiment, the body temperature of the guinea pig was maintained at 37_. Measurements of pulmonary cardiac function were carried out as previously described (Hsu T H, Lai Y L, and Kou Y R (1998) Acetylcholine and tachykinin receptor antagonists attenuate wood smoke-induced bronchoconstriction in Guinea pigs. Eur J Pharmacol 360:175-183). Total lung resistance (R_{L}) and dynamic lung compliance (C_{dyn}) were measured on a breath-by-breath basis using a computer equipped with an A/D interface (DAS 1600; Buxco Electronics, Inc., Wilmington, N.C.) and software (version 1.5.7; Buxco Electronics, Inc.). Results obtained from the computer analysis were checked for accuracy by comparison with those calculated manually.

Animals were allowed to stabilize for 10 min following surgical manipulations before the test agent was administered. Intratracheal instillation was performed using a catheter placed through the tracheal tube into the bronchial system. The catheter connected to a 1-ml syringe was inserted into the trachea cannula extending 1 cm beyond the tip of the cannula for instillation of drug or vehicle. The instillation was followed by rapid intratracheal injection of 0.5 ml of air to facilitate deposition of drug or vehicle into the lungs as in the modified methods of Chung et al. (2001)(Chung W H, Bennett B M, Racz W J, Brien J F, and TGF-beta 1 in Fischer 344 rats during aminodarone-induced pulmonary fibrosis. Am J Physiol 281:L1180-L1188). The animals were pretreated intratracheally with KMUP-3 (0.5, 1.0, and 2.0 mg/kg/150 _l of saline) or an equivalent volume (150 _l) of vehicle 15 min before TNF-_(0.01 mg/kg/300 _l saline) was instilled to induce acute bronchoconstriction. In another experiments, KMUP-3 or KMUP-1 (1.0 mg/kg i.v.) were used to compare the effects on respiratory performance, indicated by changes of tidal volume, total R_{L}, and C_{dyn}.

Isolated Tracheal preparation and Measurement of Tension: Guinea pigs (350-450 g) were anesthetized with pentobarbital (70 mg/kg) i.p. and killed by a sharp blow on the neck, followed by cervical dislocation. The trachea was excised, cleaned of adhering fat and connective tissue, cut transversely into four to five rings, and then opened by cutting longitudinally through the cartilage rings diametrically opposite the tracheal smooth muscle (Hwang T L, Wu C C, and Teng C M (1999) YC-1 potentiates nitric oxide-induced relaxation in guinea-pig trachea. Br J Pharmacol 128:577-584). Then trachea rings were suspended in a 10-ml organ bath containing Krebs' solution (118 mM NaCl, 25 mM NaHCO₃, 4.7 mM KCl, 1.2 mM KH₂PO₄, 1.2 mM MgSO₄, 11 mM glucose, and 2.5 mM CaCl₂, pH 7.3-7.4) maintained at 37_and aerated with 95% O₂ and 5% CO₂. Each tracheal tissue sample was subjected to 2 g of initial basal tension. All experiments were carried out in the presence of indomethacin (3 _M) and propranolol (1 _M) to prevent the formation of prostanoids and to inhibit _-adrenergic responses, respectively. Isometric tension was recorded with a force displacement transducer (model FT03; Grass Instruments, Quincy, M A). Before the start of measurements, all preparations were allowed to equilibrate with frequent washing for 1 h. They were first contracted with carbachol (1 _M) to determine the contractility of preparations. This was also done at the end of each experiment. The preparations were then washed and allowed to equilibrate with Krebs' solution for 50 min before being contracted a second time with carbachol (1 _M). When stable constriction to carbachol was reached, concentration responses of KMUP-3 (10, 30, and 100 _M) were obtained. Data were expressed as percentages of the maximum contractile response to carbachol (1 _M). In comparison with KMUP-3, theophylline, 3-isobutyl-1-methylxanthine (IBMX), milrinone, rolipram, zaprinast, and levcromakalim, respectively, were examined at 100 _M.

In experiments to examine the role of epithelium in tracheal relaxation, the epithelial cells were removed mechanically by rubbing the internal surface of trachea with a fine silver wire, and the removal of the epithelial layer was confirmed by histological examination as previously described (Wu et al., 2004).

To examine the possible mechanism of tracheal relaxant effects of KMUP-3, the tracheal strips were pretreated with a K⁺ channel blocker, tetraethylammonium (TEA; 10 mM); a K_{ATP} channel blocker, glibenclamide (1-M); voltage-dependent K⁺ channel blocker, 4-aminopyridine (4-AP, 100 _M); Ca²⁺-dependent K⁺ channel blockers, apamin (1 _M) and charybdotoxin (ChTX; 0.1 _M); a sGC inhibitor, 1H-[1,2,4]oxadiazolo[4,3-a]quinoxalin-1-one (ODQ; 1 _M); an NOS inhibitor, N—nitro-L-arginine methyl ester (L-NAME; 100 _M); and an adenylate cyclase (AC) inhibitor, SQ 22536 (100 _M) for 30 min prior to the addition of KMUP-3 (10, 30, and 100 _M). In the other experiments, TSMs were precontracted with 60 mM KCl to examine the relaxation achieved by KMUP-3. The KCl solution was prepared by substituting NaCl with KCl (60 mM) in an equimolar amount.

Determination of Nitrite: To measure concentrations of nitrite (NO₂⁻) in guinea pig TSM strips, TSM strips were pretreated with KMUP-3 (100 _M) in Krebs' solution for 30 min. Then guinea pig TSM strips (2 ml/g tissue of methanol) were ultrasonically homogenized in an ice bath and then centrifuged at 10,000 g for 10 min at 4_. Briefly, harvestable supernatants were reacted with an equal volume of Griess reagents (1% sulfonamide/0.1% N-L-naphthylethylenediamine dihydrochloride/5% H₃PO₄) for 10 min to form a colored azo dye. The absorbance at 540 nm was detected by a flow-through visible spectrophotometer, and an equal volume of methanol was used for blank control.

Cell Culture of Tracheal Smooth Muscle: Guinea pigs (350-450 g) were injected i.p. with a lethal dose of pentobarbital. The tracheas were excised and cut longitudinally through the cartilage. Using a dissecting microscope, TSM strips were dissected from the surrounding parentchyma. The epithelium was removed from the luminal surface, and bands of TSM were gently separated from the underlying connective tissue. Then TSM strips were chopped into small sections (1 mm³) and incubated in Hanks' balanced salt solution (138 mM NaCl, 4 mM NaHCO₃, 5 mM KCl, 0.3 mM KH₂PO₄, 0.3 mM Na₂HPO₄, and 1.0 mM glucose) with 0.05% elastase type IV and 0.2% collagenase type IV (Invitrogen, Carlsbad, Calif.) for 30 min at 37_ with gentle shaking. The solution of dissociated smooth muscle cells was centrifuged (6 min at 500 g), and the pellet was resuspended in 1:1 Dulbecco's modified Eagle's medium/Ham's F-12 medium supplemented with 10% fetal bovine serum, 0.244% NaHCO₃, and 1% penicillin/streptomycin. Cells were cultured in 25-cm² flasks at 37_ in humidified air containing 5% CO₂. Confluent cells were detached with 0.25% trypsin-0.02% EDTA at 37_and then subcultured to establish secondary cultures. Cultures were maintained for no more than four passages. They were identified as smooth muscle cells by the typical hill-and-valley appearance, and cellular homogeneity was further confirmed by the presence of smooth muscle specific _-myosin and _-actin immunoreactivity. Indirect immunofluorescence staining for a variety of antigens was carried out by first plating the cells on chamber slides, fixing the cells in 3.7% formaldehyde-phosphate-buffered saline for 10 min, and permeabilizing the cells with phosphate-buffered saline and 0.1% Triton X-100. Cells were then stained with either a mouse monoclonal antibody directed against the amino-terminal 10 amino acids of _-smooth muscle actin or _-myosin (Roche Diagnostics, Indianapolis, Ind.). All cells were stained with fluorescein-labeled goat anti-mouse IgG antibody (Roche Diagnostics). Over 95% of the cell preparation was found to be composed of smooth muscle cells.

Phosphodiesterase Activity: PDE activity was determined by the method of Nicholson et al. (Nicholson C D, Challiss R A, and Shahid M (1991) Differential modulation of tissue function and therapeutic potential of selective inhibitors of cyclic nucleotide phosphodiesterase isonzymes. Trends Pharmaclo Sci 12:19-27). PDE3 and PDE5 purified from human platelets and PDE4 purified from human U-937 pronocytic cells were used. Test compound (10 _M) or vehicle was incubated with 0.2 _g of enzyme and 1 _M cAMP containing 0.01 _M [³H]cAMP in Tris buffer, pH 7.5, for 20 min at 25_. The reaction was terminated by boiling for 2 min, and the resulting cAMP was converted to adenosine by the addition of 10 mg/ml snake venom nucleotidase, which is followed by a further incubation at 37_for 10 min. Unhydrolyzed cAMP was bound to AG1-X2 resin, and the remaining [³H]adenosine in the aqueous phase was quantitated by scintillation counting.

Another test compound (10 _M) or vehicle was incubated with 3.5 _g of enzyme and 1 _M cGMP containing 0.01 _M [³H]cGMP in Tris buffer, pH 7.5, for 20 min at 25_. The reaction was terminated by boiling for 2 min, and the resulting GMP was converted to adenosine by the addition of 10 mg/ml snake venom nucleotidase, which was followed by a further incubation at 37_ for 10 min. Unhydrolyzed cGMP was bound to AG1-X2 resin, and the remaining [³H]guanosine in the aqueous phase was quantitated by scintillation counting.

Measurement of cAMP and cGMP: Intracellular concentrations of cAMP and cGMP in TSMCs were measured as in our previous reports (Wu B N, Lin R J, Lin C Y, Shen K P, Chiang L C, and Chen I J (2001) A xanthine-based KMUP-1 with cyclic GMP enhancing and K⁺ channels opening activities in rat aortic smooth muscle. Br J Pharmacol 134:265-274; Lin R J, Wu B N, Lo Y C, Shen K P, Lin Y T, Huang C H, and Chen I J (2002) KMUP-1 relaxes rabbit corpus cavernosum smooth muscle in vitro and in vivo: involvement of cyclic GMP and K(+) channels, Br J Pharmacol 135:1159-1166). In brief, cells were finally grown in 24-well plates (10⁵ cells/well). At confluence, monolayer cells were washed with phosphate-buffered saline and then incubated with KMUP-3 (0.01-100 _M) for 20 min at 37_ in medium. Milrinone, rolipram, zaprinast, theophylline, or aminophylline (100 M) was added for 20 min. The reaction was terminated by replacement of medium with 1 ml of ice-cold 1 N hydrochloric acid. Cells suspensions were sonicated and then centrifuged at 2500 g for 15 min at 4_. Then, the supernatants were lyophilized and the cAMP and cGMP of each sample were determined using commercially available radioimmunoassay kits (GE Healthcare, Little Chalfont, Buckinghamshire, UK).

Expression of PKA and PKG: The expression of PKA and PKG was determined by Western blot as previously described by Wu et al. and Murthy et al. (Wu B N, Lin R J, Lo Y C, Shen K P, Wang C C, Lin Y T, and Chen I J (2004) KMUP-1, a xanthine derivatives, induces relaxation of guinea-pig isolated trachea: the role of the epithelium, cyclic nucleotides and K⁺ channels. Br J Pharmacol 142:1105-1114; Murthy K S, Zhou H, and Makhlouf G M (2002) PKA-dependent activation of PDE3A and PDE4 and inhibition of adenylyl cyclase V/VI in smooth muscle. Am J Physiol 282:C508-C517). Guinea pig TSMCs were incubated with KMUP-3 for 30 min. Each of the cell lysates, each containing 1 _g of cellular protein, was electrophoresed in 7.5% SDS-polyacrylamide gel electrophoresis and then transferred to polyvinylidene diflouride membrane (Millipore Corporation, Billerica, Mass.). The membrane was stained with Ponceau S to verify the integrity of the transferred proteins and to monitor the unbiased transfer of all protein samples. The membrane was then washed with 25 ml of TBS (100 mM NaCl, 0.1% and 10 mM Tris-HCl, pH 7.5) for 5 min at room temperature and incubated in 25 ml of blocking buffer (TBS plus 0.1% Tween 20 and 5% nonfat milk) overnight at 4_. In the measurement of protein, the membrane was incubated with appropriate PKA_{RI} or PKA₁_{_{—}}₁_{_{—}} primary antibody (diluted 1:250) in 2 ml of blocking buffer for 1 h at room temperature, washed three times for 5 min each with 15 ml of Tris-buffered saline/Tween 20 (TBS plus 0.1% Tween 20), incubated with horseradish peroxidase-conjugated secondary antibody (1:10,000) in 15 ml of blocking buffer with gentle agitation for 1 h at room temperature, and finally washed three times for 5 min each time with 15 ml of Tris-buffered saline/Tween 20. The membrane was then subjected to enhanced chemiluminescence (GE Healthcare) for the detection of the specific antigen.

Drugs and Chemicals: Leveromakalim was generously supplied by the GlaxoSmithKline (Uxbrige, Middlesex, UK) and dissolved in ethanol at 10 mM. 4-AP, aminophylline, apamin, carbachol, ChTX, forskolin, glibenclamide, Griess reagents, IBMX, indomethacin, Krebs' solution reagents, L-NAME, methylene blue, ODQ propranolol, SQ 22536, TEA, theophylline, and TNF-_ were all obtained from Sigma-Aldrich (St. Louis, Mo.). PKA_{RI} or PKA₁_{_{—}}₁_{_{—}} antibodies were purchased from Calbiochem (San Diego, Calif.). All drugs and reagents were dissolved in 0.05 M acetic acid; indomethacin was dissolved in 100 mM sodium carbonate; ChTX, glibenclamide, milrinone, rolipram, zaprinast, IBMX, and ODQ were dissolved in DMSO at 10 mM and serially diluted with distilled water; and KMUP-3, synthesized in our laboratory, was dissolved in 10% absolute alcohol, 10% propylene glycol, and 2% 1N HCl at 10 mM. Serial dilutions were made in distilled water.

Statistical Evaluation of Data: The results are expressed as mean±S.E. Statistical differences were determined by independent and paired Student's t test in unpaired and paired samples, respectively. Whenever a control group was compared with more than one treated group, the one-way ANOVA or two-way repeated measures ANOVA was used. When the ANOVA manifested a statistical difference, the Dunnett's or Student-Newman-Keuls test was applied. P_{—}0.05 was considered to be significant in all experiments. Analysis of the data and plotting of the figures were performed with the aid of software (SigmaPlot version 8.0 and SigmaStat Version 2.03, Chicago, Ill.) run on an IBM-compatible computer.

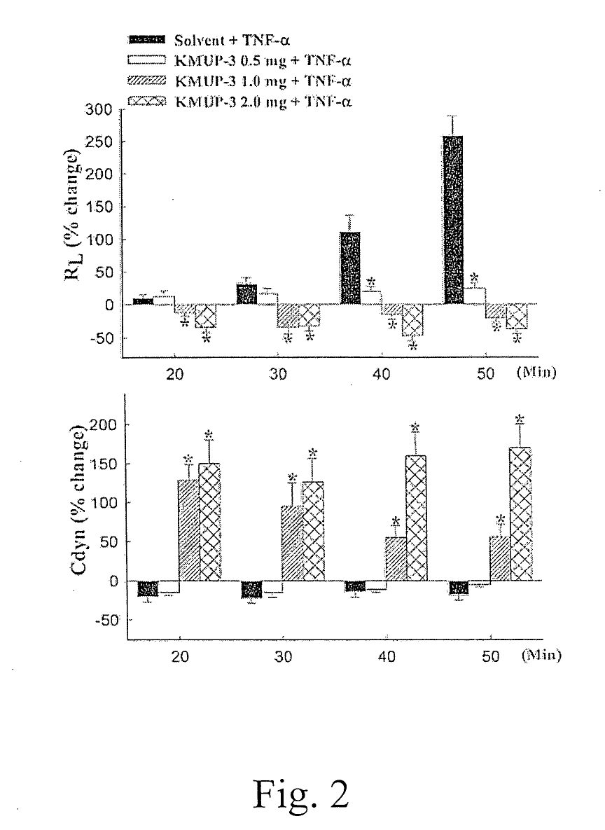

Pulmonary Function: The baseline values of total lung resistance, dynamic lung compliance, and tidal volume were 0.25±0.02 cmH₂O ml⁻¹s⁻¹, 0.31±0.02 ml cmH₂O⁻¹, and 1.91±0.11 ml, respectively. TNF-_-induced bronchocontraction by intratracheal instillation increases R_{L} and decreases C_{dyn}, respectively (Please see FIG. 2). KMUP-3 (0.5, 1.0, and 2.0 mg/kg) instilled through trachea for 15 min prior to TNF-_-induced bronchocontraction could reverse the situation by decreasing R_{L} and by increasing C_{dyn} (Please see FIG. 2). Blood pressure and heart rate were not significantly changed by instillation of KMUP-3 during the recording of lung function. In another experiment, i.v. KMUP-3 (1 mg/kg) more significantly decreases R_{L} and increased both C_{dyn} and respiratory tidal volume than KMUP-1 (Table 1).

TABLE 1
Effects of KMUP-3 and KMUP-1 on PDE inhibitory activity and lung
resistance, dynamic compliance, and tidal volume maximum changes
The enzyme inhibitory activity was measured by 10 _M KMUP-1 and KMUP-3
(n = 3) on various PDEs, R_{L}, C_{dyn}, and tidal volume were measured after i.v.
injection of KMUP-1 or KMUP-3 (1 mg/kg)

| Compound | PDE3 inhibition | PDE4 Inhibition | PDE5 Inhibition | R_{L} | C_{dyn} | Tidal Volume |
|---|---|---|---|---|---|---|
| % | | | | Maximum change of baseline (%) | | |
| KMUP-1 | 23 ± 2.2 | 18 ± 2.3 | 29 ± 2.5 | −20 ± 3.5 | 35 ± 4.5 | 2.8 ± 0.2 |
| KMUP-3 | 55 ± 2.5* | 48 ± 2.1* | 48 ± 1.9* | −55 ± 4.5* | 50 ± 5.5* | 21.7 ± 0.5* |

*P _ 0.05 compared with KMUP-1, respectively (n = 3).

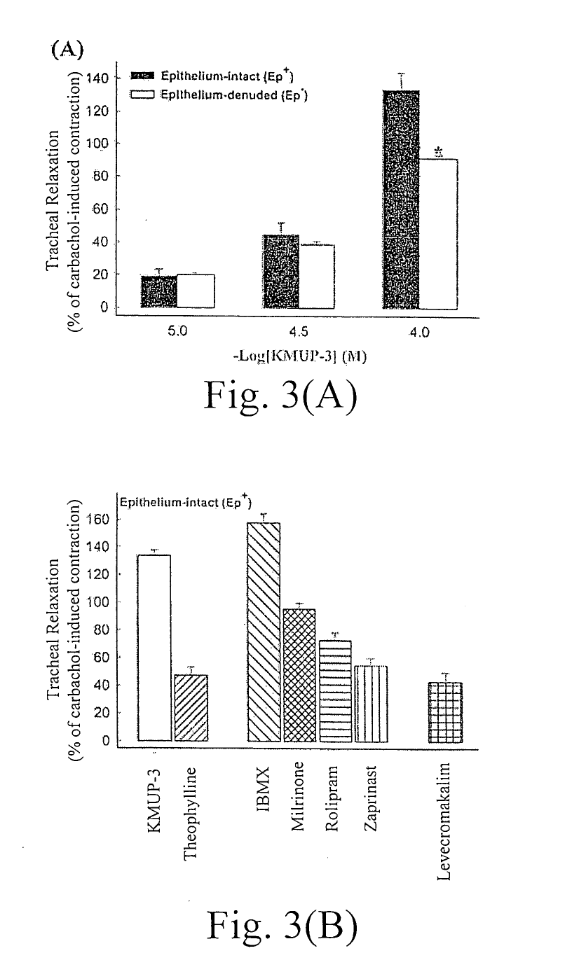

NOS and Epithelium-Derived Relaxation: As shown in FIG. 3(A), KMUP-3 (10, 30, and 100 _M) produced concentration-dependent relaxations both in epithelium-denuded and epithelium-intact (E_{P}⁺) guinea pig, indicating that KMUP-3 induced epithelium-independent relaxation activity. However, KMUP-3 did demonstrate a significant decrease in the relaxation response after epithelium denudation, suggesting that at least part of the observed effect is epithelium-dependent. Obviously, KMUP-3 completely relaxed the TSM strips at 100 _M, achieving even up to 130% relaxation. In addition, the relaxations of guinea pig E_{P}⁺ TSM elicited by KMUP-3 were significantly inhibited by pre-treatment with an NOS inhibitor L-NAME. The NO₂— release reached 160±15% (n=3) by KMUP-3 (100 _M) in comparison with the basal level (0.8±0.1 _M/mg) in E_{P}⁺ guinea peg TSM.

KMUP-3, theophylline, IBMX, milrinone, rolipram, and zaprinast (100 _M) were all found to cause relaxations in epithelium-intact trachea. Both IBMX and KMUP-3 were more potent at 100 _M concentrations than theophylline-inducing tracheal relaxation (Please see FIG. 3(B)). A potassium channel opener, levcromakalim (100 _M), also displayed relaxation effects on TSM (E_{P}⁺)(Please see FIG. 3(B)).

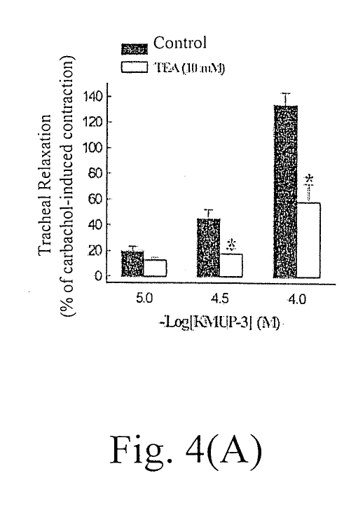

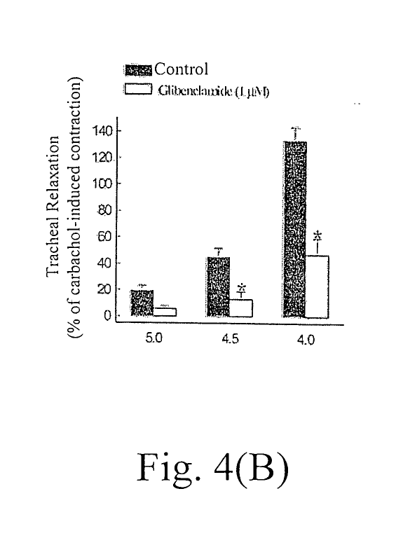

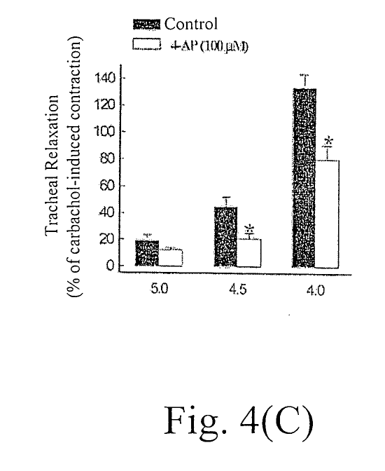

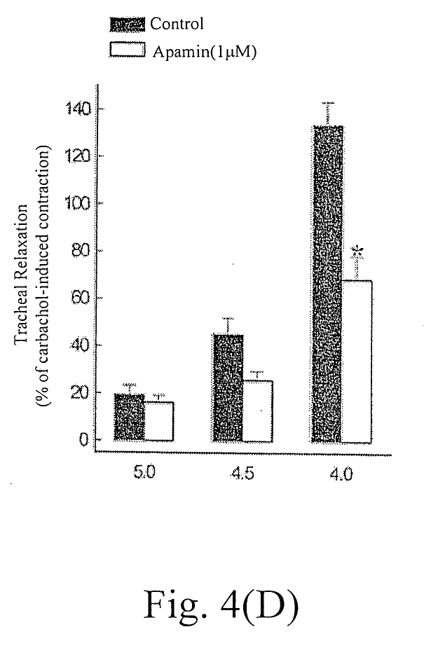

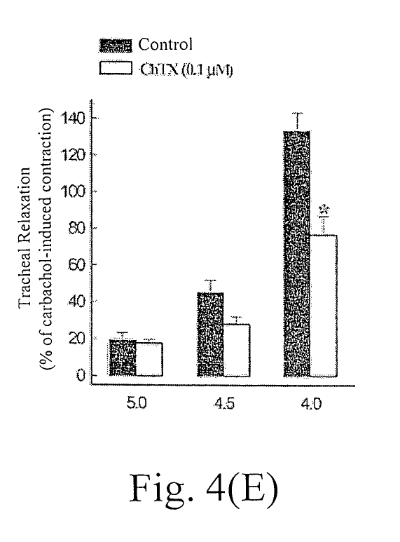

K⁺ Channel Activities: KMUP-3 (10, 30, and 100 _M) caused a concentration-dependent relaxation in carbachol- or high-K⁺-contracted guinea pig E_{P}⁺ TSM. KMUP-3 against carbachol-induced contractions in TSM (E_{P}⁺) was inhibited by the K⁺ channel blocker, TEA; a K_{ATP} channel blocker, glibenclamide; a voltage-dependent K⁺ channel blocker, 4-AP; and Ca²⁺-dependent K⁺ channel blockers, apamin and ChTX (Please see FIGS. 4(A)-4(E)).

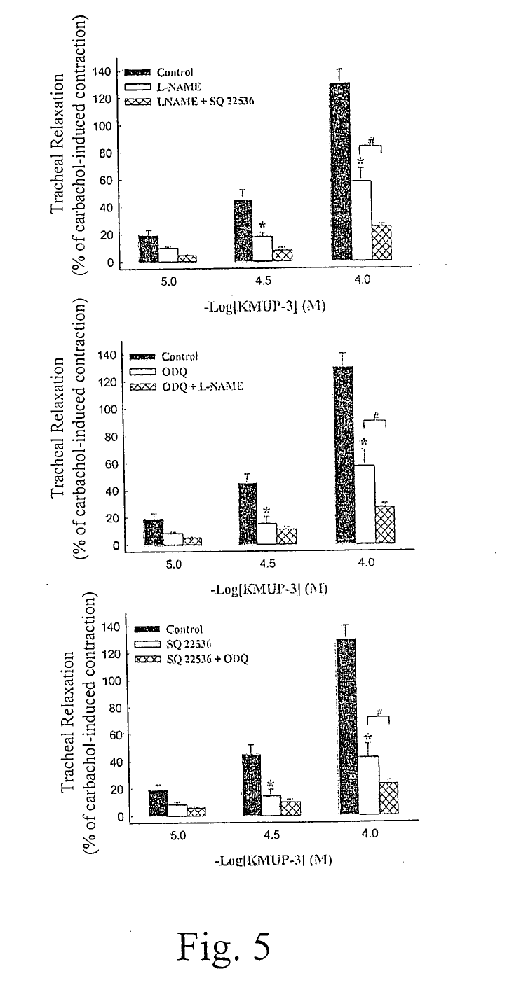

Involvement of sGC and Adenylate Cyclase: The relaxations of guinea pig E_{P}⁺ TSM elicited by KMUP-3 were attenuated by pretreatment with sGC inhibitor, ODQ (1 _M); an NOS inhibitor, L-NAME (100 _M); or an adenylate cyclase inhibitor, SQ 22536 (100 _M)(Please see FIG. 5). Additionally, KMUP-3-induced relaxations were dramatically reduced by the pretreatment with L-NAME+SQ 22536, ODQ+L-NAME, or SQ 22536+ODQ (Please see FIG. 5).

Inhibition of Phosphodiesterase: The enzyme inhibitory activity of KMUP-3 on PDEs was measured and compared with KMUP-1. As shown in Table 1, KMUP-3 displays different inhibition activities on PDE3, PDE4, and PDE5. The result indicated that the enzyme inhibition activity of KMUP-3 appears to be more potent than KMUP-1, but it has no selectivity among PDE3, 4, or 5. We also observed that the inhibitory effects of theophylline (10_M) on PDE3, 4, and 5 were respectively 8±1.2, 7.9±1.2, and 12±1.5% (n=3). The IC₅₀ values of KMUP-3 for PDE3, PDE4, and PDE5 were 8.5±1.5, 14.3±2.2, and 14.5±1.8_M (n=3), respectively, whereas KMUP-1's IC₅₀ values for those PDEs were greater than 100 _M. Under this condition, IBMX was used as a reference agent, and its IC₅₀ values for PDE3, PDE4, and PDE5 were 6.4±1.6 25.6±4.9, and 30.8±4.5_M (n=3), respectively.

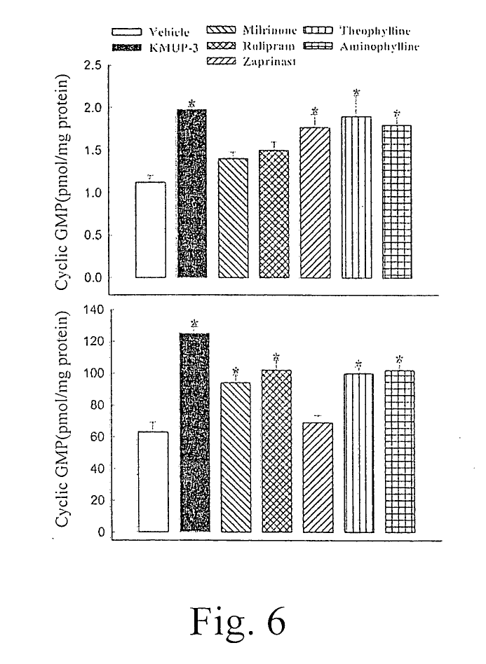

Accumulation of cAMP and cGMP: cAMP and cGMP levels were examined in guinea pig primary TSMCs. The amounts of basal levels of cAMP and cGMP in the cells were 62.6±6.4 and 1.18±0.06 pmol/mg protein, respectively (n=3). KMUP-3 (0.01, 0.1, 1, 10, and 100 _M) significantly increased both cAMP (80.2±2.5, 91.3±3.4, 97.9±2.5, 110.7±4.5, and 127.5±4.8 pmol/mg protein) and GMP(1.62±0.05, 1.73±0.01, 1.75±0.04, 1.86±0.05, and 1.98±0.09 pmol/mg protein) levels compared with each basal value in guinea pig primary TSMCs. Moreover, we compared both cAMP and GMP levels of KMUP-3 with theophylline, aminophylline, milrinone, rolipram, and zaprinast at 100 _M (Please see FIG. 6). KMUP-3, theophylline, aminophylline, and zaprinast significantly enhances the amounts of cGMP, but this was not observed in milrinone or rolipram for selective PDE3 and PDE4 inhibitions, respectively. On the other hand, KMUP-3, theophylline, aminophylline, milrinone, and rolipram all elicited significant elevations of cAMP accumulation, but this was not so for the selective PDE5 inhibitor zaprinast (Please see FIG. 6). The elevated cAMP levels induced by KMUP-3 and forskolin (100 _M), an adenylate cyclase activator, were inhibited by the pretreatment with the adenylate cyclase inhibitor, SQ 22536 (100 _M)(Please see Table 2). Correspondingly, the rise of cGMP accumulation by KMUP-3 was abolished by the pretreatment with the NOS inhibitor L-NAME (100 _M) or the sGC inhibitor ODQ (10 _M), respectively (Please see Table 2).

TABLE 2
Effects of KMUP-3 and forskolin (100 _M) on cAMP and cGMP levels in
TSMCs in the absence and presence of SQ 22536 (100 _M), L-NAME
(100 _M), or ODQ (10 _M)
Each value represents the mean ± S.E.M. of
three independent experiments.
Treatment
cAMP (pmol/mg protein)

| | |
|---|---|
| Vehicle | 62.60 ± 6.40 |
| SQ 22536 | 74.32 ± 7.56 |
| Forskolin | 119.15 ± 5.11* |
| Forskolin plus SQ 22536 | 94.33 ± 3.42** |
| KMUP-3 | 128.72 ± 4.57* |
| KMUP-3 plus SQ 22536 | 74.51 ± 3.18** |

Cyclic GMP (pmol/mg protein)

| | |
|---|---|
| Vehicle | 1.18 ± 0.06 |
| KMUP-3 | 1.98 ± 0.08* |
| L-NAME | 1.12 ± 0.02 |
| KMUP-3 plus L-NAME | 1.28 ± 0.01** |
| ODQ | 1.12 ± 0.03 |
| KMUP-3 plus ODQ | 1.35 ± 0.04** |

*P _ 0.05 compared with the vehicle, ANOVA followed by Dunnett's test
**P _ 0.05 compared with forskolin or KMUP-3

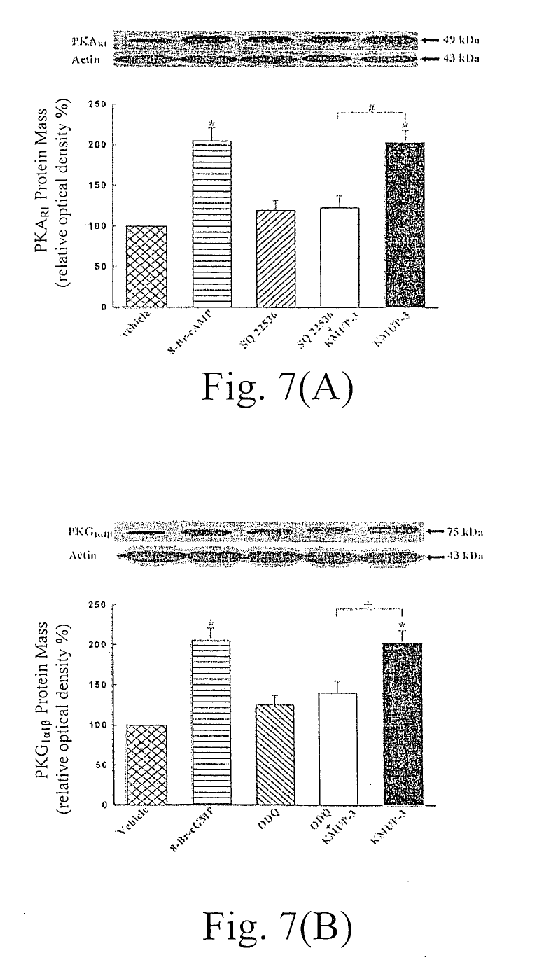

Expression of PKA and PKG: Expression of immunoreactive PKA_{RI} is shown in FIG. 7(A). Monoclonal antibody to PKA_{RI} recognized a band at 49 kDa in extracts of tracheal smooth muscle. KMUP-3 (1 _M) stimulated the expression of immunoreactive PKA_{RI} protein. Treatment with SQ 22536 (100 _M) for 30 min prior to KMUP-3 (1 _M) attenuated the expression of PKA_{RI} (Please see FIG. 7(A)). Both 8-Br-cAMP and KMUP-3 (1 _M) increased the expression of PKA_{RI}; however, pretreatment with ODQ (10 _M) for 30 min prior to KMUP-3 did not significantly affect the PKA protein expression by KMUP-3 (B. N. Wu and I. J. Chen, unpublished data). Expression of immunoreactive PKA₁_{_{—}}₁_{_{—}} is shown in FIG. 7(B). Polyclonal antibody to PKA₁_{_{—}}₁_{_{—}} recognized a band at 75 kDa in extracts of tracheal smooth muscle. Both 8-Br-cGMP and KMUP-3 (1 _M) increased the expression of PKA₁_{_{—}}₁_{_{—}} and pretreatment with ODQ (10_M) for 30 min prior to KMUP-3 (1 _M) significantly reduced the PKG protein induced by KMUP-3 (Please see FIG. 7(B)).

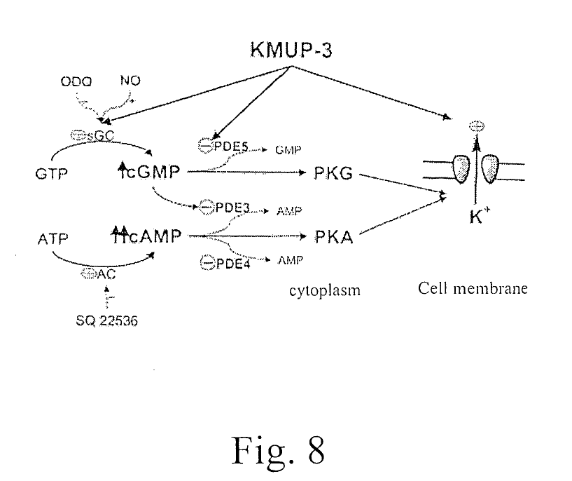

Relaxation of smooth muscles are through activation of AC and sGC and accumulation of cAMP and cGMP (Ignarro L J and Kadowitz P J (1985) The pharmacological and physiological role of cyclic GMP in vascular smooth muscle relaxation. Annu Rev Pharmacol Toxicol 25:171-191). cAMP and cGMP are degraded by PDE, which catalyzes the conversion of cAMP to 5′-AMP and cGMP to 5′-GMP and therefore leads to decreases in intracellular cAMP or cGMP levels. Among them, PDE3 is inhibited by cGMP, indirectly leading to the increase of cAMP. Accumulations of cAMP and cGMP further induced the increases of PKG and PKA. In this application, KMUP-3 significantly increases the expression of PKA and PKG and thus confirms the contents of cAMP and GMP in TSMCs. The rank order of relaxant activity at 100 _M was KMUP-3_KMUP-1 (Wu B N, Lin R J, Lo Y C, Shen K P, Wang C C, Lin Y T, and Chen I J (2004) KMUP-1, a xanthine derivative, induces relaxation of guinea-pig isolated trachea: the role of the epithelium, cyclic nucleotides and K⁺ channels. Br J Pharmacol 142:1105-1114)_IBMX_milrinone_rolipram_zaprinast, theophylline, and levcromakalim (Please see FIG. 3(B)). This result is adaptable to enzyme inhibition activity and IC₅₀ of KMUP-3 pm PDE3, 4, and 5, in comparison with previous KMUP-1. We suggest that the higher inhibition (percentage) of KMUP-3 than KMUP-1 on PDE3, 4, and 5 favors better respiratory performance as shown by increased respiratory tidal volume, decreased R_{L}, and increased C_{dyn} (Table 1). In this application, KMUP-3 is further suggested to have PDE inhibition and action of epithelium-derived NO to increase cGMP and cAMP. KMUP-3 retains partial PDE4 inhibition activity, which increases cAMP, and subsequently the expression of PKA in TSMs. Inhibition of PDE5 activity by KMUP-3 may increase cGMP, which has proven to inhibit PDE3 activity, resulting in increase of cAMP, and subsequently the expression of PKG and partial PKA (Please see FIG. 8).

KMUP-3 is suggested to accumulate cAMP/cGMP and enhance K⁺ efflux, leading to reduction of Ca²⁺ influx-associated contractility in TSM (Please see FIG. 8). Whether accumulation of cAMP or cGMP caused by KMUP-3 is attributable to K⁺ efflux in TSMC remains to be resolved. In this application, the expression of PKG₁_{_{—}}₁_{_{—}} and PKA_{RI}, able to be activated by cGMP/cAMP, was suggested to mediate the K⁺ channel opening activity through their cGMP/cAMP-increasing activities.

The combination of the multiple actions of KMUP-3 above thus may contribute to significant relaxation of carbachol-induced TSM contraction in vitro and protect against TNF-_-induced increases of R_{L} and decreases of C_{dyn} in vivo. It is suggested that PDE inhibition, cAMP/cGMP increasing, and K⁺ channel activities of KMUP-3 are the crucial determinants for its relaxation effects on TSM. Particularly, more PDE4-inhibiting activity by KMUP-3 than by KMUP-1 may favor the former as useful in increasing respiratory performance.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A nitrophenylpiperazine derivative of xanthine being one of a substrate with the formula I and a pharmaceutically acceptable salt thereof and having an ability of relaxing airway smooth muscle 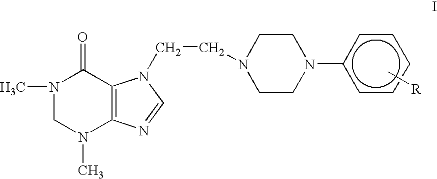
wherein R is a nitroso group.

2. The nitrophenylpiperazine derivative of xanthine according to claim 1, having an effect of tracheal relaxation.

3. The nitrophenylpiperazine derivative of xanthine according to claim 1, having an effect of anti-airway constriction induced by a tumor necrosis factor (TNF)-_.

4. The nitrophenylpiperazine derivative of xanthine according to claim 1, providing the benefits for the treatment of asthma and COPD (chronic obstructive pulmonary disease).

5. A pharmaceutical composition for relaxing airway smooth muscle comprising a nitrophenylpiperazine derivative of xanthine of claim 1 and a pharmaceutically acceptable carrier.

6. The pharmaceutical composition according to claim 4, further comprising an excipient.

7. A method of manufacturing a medicament for relaxing airway smooth muscle comprising providing one of a nitrophenylpiperazine derivative of xanthine having the formula I and a pharmaceutically acceptable salt thereof as a raw material 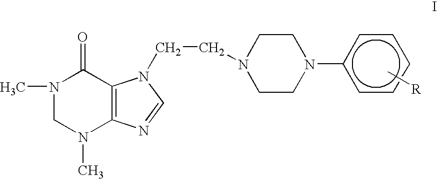
wherein R is a nitroso group.

8. A use of a nitrophenylpiperazine derivative of claim 1 for manufacturing a medicament having an ability of relaxing airway smooth muscle.
